(19) United States
(12) Patent Application Publication
(10) Pub. No.: US 2024/0382247 A1
(43) Pub. Date: Nov. 21, 2024

(54) SYSTEMS AND METHODS FOR TISSUE ABLATION

(71) Applicant: Medtronic Xomed LLC, Minneapolis, MN (US)

(72) Inventors: Nicholas F. Pergola, Lafayette, CO (US); James Britton Hissong, Jacksonville, FL (US)

(21) Appl. No.: 18/633,671

(22) Filed: Apr. 12, 2024

Related U.S. Application Data
(60) Provisional application No. 63/467,363, filed on May 18, 2023.

(51) Int. Cl. A61B 18/14; A61B 17/00; A61B 18/00

(52) U.S. Cl. CPC A61B 18/1482; A61B 2017/00128; A61B 2018/00577; A61B 2018/00642; A61B 2018/00994

(57) ABSTRACT

An ablation system includes an ablation device having an electrode configured to deliver electrosurgical energy to tissue and an electrosurgical generator configured to deliver electrosurgical energy to the electrode. The electrosurgical generator is also configured to calculate a number of Joules of electrosurgical energy delivered to the electrode. The ablation system also includes a feedback device. The feedback device is configured to provide feedback (e.g., audible and/or visual) correlated with the calculated number of Joules of electrosurgical energy delivered to the electrode.

### CROSS-REFERENCE TO RELATED APPLICATIONS

This application claims the benefit of the filing date of provisional U.S. Patent Application No. 63/467,363 filed on May 18, 2023.

### FIELD

The disclosure relates to tissue ablation. More specifically, the disclosure relates to ablation of tissue using a “moving shot” technique.

### BACKGROUND

Conventional approaches to treating benign thyroid nodules and malignant thyroid cancers include removal of these tissues under general anesthesia by making an incision in the neck and resecting the tissues. However, aside from the inherent risks of undergoing general anesthesia, the patient also risks the possibility of undesirable side effects such as a residual neck scar, nerve damage, hypothyroid function, etc.

Improved approaches to reducing benign thyroid nodules and some malignant cancerous tumors involve percutaneously inserting, under the guidance of ultrasound imaging, a radio frequency (RF) ablation device under local anesthesia and ablating target tissue. Generally, tissue ablation involves inserting an ablation electrode (e.g., RF, microwave, or laser fiber) into target tissue and monitoring energy delivered into the target tissue to achieve a desired tissue temperature for a given period of time when using a traditional static ablation technique, or by producing a vigorous ablation environment using higher energy levels and moving the electrode through the tissue while ablating, which is known as a moving-shot technique. Using an ultrasound system including a suitable display, ablation of the target tissue can be observed such that the nodule is visible and the electrode can be observed percutaneously entering the surgical site and ablating the target tissue as the visualized target tissue changes in appearance due to heating of the target tissue caused by delivery of ablation energy to the target tissue. Using a traditional static ablation technique, the electrode is held in place to achieve a desired ablation zone and then can be moved to a new position and the process repeated until therapy for a target region is complete. In contrast to the traditional static ablation technique, the moving-shot technique involves delivering an increased energy level to tissue (e.g., increased relative to an energy level used in a traditional static ablation technique) to produce vigorous ablation and the electrode is moved either continuously through tissue or moved using a start-stop technique where the electrode is briefly held in place (e.g., held in place for a shorter amount of time relative to the above-noted traditional static ablation technique), moved to a new position, and the process repeated until therapy for a target region is complete. In some instances, this process is moderated by a specific parameter such as, for example, tissue temperature and/or tissue impedance, which is controlled by energy delivery to the target tissue and feedback from the ablation device to the generator.

### SUMMARY

Provided in accordance with aspects of the present disclosure is an ablation system including an ablation device configured to be inserted into an ablation target. The ablation device has an electrode configured to be placed within the ablation target and retracted along an axis defined through the ablation target while delivering energy to tissue. The ablation system also includes an electrosurgical generator configured to deliver electrosurgical energy to the ablation device and to calculate a number of Joules of electrosurgical energy delivered to the electrode. The ablation system also includes a display device and an audio device. The display device is in communication with the electrosurgical generator and is configured to display the calculated number of Joules of electrosurgical energy delivered to the electrode during an ablation procedure. The audio device is in communication with the electrosurgical generator and is configured to broadcast a sound correlated with the number of Joules of electrosurgical energy delivered to the electrode such that the audio device changes at least one property of the sound based on the calculated number of Joules of energy delivered to the electrode.

In an aspect of the present disclosure, the electrosurgical generator is configured to calculate a suggested total number of Joules of electrosurgical energy for delivering to the electrode based on at least one of a size of the ablation target or a size of the electrode.

In another aspect of the present disclosure, the display device is configured to display the suggested total number of Joules of electrosurgical energy for delivering to the electrode.

In another aspect of the present disclosure, the electrosurgical generator is configured to receive as input a total number of Joules of electrosurgical energy for delivering to the electrode.

In another aspect of the present disclosure, the audio device is configured to broadcast a sound associated with at least one of the calculated number of Joules of electrosurgical energy delivered to the electrode reaching the total number of Joules of electrosurgical energy received as input, or the calculated number of Joules of electrosurgical energy delivered to the electrode approaching the total number of Joules of electrosurgical energy received as input.

In still another aspect of the present disclosure, the display device is configured to display the input total number of Joules of electrosurgical energy received by the electrosurgical generator.

In still yet another aspect of the present disclosure, the electrosurgical generator is configured to receive as input at least one of a size of the ablation target and a size of the electrode and calculate a suggested total number of Joules of electrosurgical energy for delivering to the electrode based on the received input.

In another aspect of the present disclosure, the ablation target is a thyroid nodule.

In another aspect of the present disclosure, the audio device is integral with the electrosurgical generator.

In still another aspect of the present disclosure, the display device is integral with the electrosurgical generator.

In still another aspect of the present disclosure, the at least one property of the sound includes a volume, an intensity, a frequency, a periodicity, or a tone of the sound.

In still yet another aspect of the present disclosure, the audio device is configured to broadcast a verbal indication associated with the calculated number of Joules of electrosurgical energy delivered to the electrode.

Also provided in accordance with the present disclosure is a method for ablating a thyroid nodule. The method includes inserting an electrode of an ablation device into a first portion of a thyroid nodule along an axis defined through the thyroid nodule and delivering electrosurgical energy from the electrode to the thyroid nodule while retracting the electrode along the axis toward a second portion of the thyroid nodule. The method also includes calculating a number of Joules of electrosurgical energy delivered to the electrode and displaying the calculated number of Joules of electrosurgical energy delivered to the electrode. The method also includes broadcasting a sound correlated with the number of Joules of electrosurgical energy delivered to the electrode, controlling at least one property of the sound based on the calculated number of Joules of electrosurgical energy delivered to the electrode, and repeating the previous steps for at least one additional axis defined through the thyroid nodule until the ablation procedure is complete.

In an aspect of the present disclosure, the method also includes receiving as input at least one of a size of the thyroid nodule or a size of the electrode and calculating a suggested total number of Joules of electrosurgical energy for delivering to the electrode based on the received input.

In another aspect of the present disclosure, the method also includes displaying the suggested total number of Joules of electrosurgical energy for delivering to the electrode.

In still another aspect of the present disclosure, the method also includes receiving as input a total number of Joules of electrosurgical energy for delivering to the electrode.

In yet another aspect of the present disclosure, the method also includes broadcasting a sound associated with at least one of the calculated number of Joules of electrosurgical energy delivered to the electrode reaching the total number of Joules of electrosurgical energy received as input, or the calculated number of Joules of electrosurgical energy delivered to the electrode approaching the total number of Joules of electrosurgical energy received as input.

In another aspect of the present disclosure, the method also includes displaying the total number of Joules of electrosurgical energy received as input.

Another ablation system is provided in accordance with the present disclosure and includes an ablation device having an electrode configured to deliver electrosurgical energy to tissue and an electrosurgical generator configured to deliver electrosurgical energy to the electrode and to calculate a number of Joules of electrosurgical energy delivered to the electrode. The ablation system also includes a feedback device. The feedback device is in communication with the electrosurgical generator and is configured to generate feedback correlated with the calculated number of Joules of electrosurgical energy delivered to the electrode relative to a set total number of Joules of electrosurgical energy.

In an aspect of the present disclosure, the feedback device is one of an audio device configured to generate audible feedback or a display device configured to generate visual feedback.

### BRIEF DESCRIPTION OF THE DRAWINGS

Aspects of the disclosure are described herein with reference to the accompanying drawings, wherein:

FIG. 1 is a schematic illustration of an ablation system including an ablation device, in accordance with aspects of the present disclosure;

FIG. 2 is a top plan view of a thyroid gland including depictions of conceptual ablation units and illustrating the ablation device of FIG. 1 inserted into a thyroid nodule for performing an ablation procedure, in accordance with aspects of the present disclosure;

FIG. 3 is a flowchart illustrating an example approach for performing an ablation procedure on a thyroid nodule using a “moving shot” technique, in accordance with aspects of the present disclosure; and

FIG. 4 is a schematic illustration of an exemplary robotic surgical system configured for use with the ablation system of FIG. 1, in accordance with aspects of the present disclosure.

### DETAILED DESCRIPTION

Embodiments of the disclosure are now described in detail with reference to the drawings in which like reference numerals designate identical or corresponding elements in each of the drawings. The aspects may be combined in any manner consistent with the functionality of the apparatus and/or method disclosed herein. As used herein, the term “clinician” refers to a doctor, a clinician, a nurse, or any other care provider and may include support personnel. Throughout this description, the term “proximal” will refer to the portion of the device or component thereof that is closer to the clinician and the term “distal” will refer to the portion of the device or component thereof that is farther from the clinician. As used herein, the term “exemplary” does not necessarily mean “preferred” and may simply refer to an example unless the context clearly indicates otherwise.

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the disclosure in unnecessary detail.

The present disclosure relates to ablation of a thyroid nodule using a “moving shot” technique. Ablation treatment of thyroid nodules and tumors typically includes inserting an ablation device at the midline of the neck in a lateral trajectory (e.g., using a trans-isthmic approach) for treatment of a nodule in either the right or left thyroid gland using a suitable form of ablation energy such as, for example, radiofrequency (RF), microwave, laser, cryogenics, or the like. Using ultrasound guidance, the insertion and placement of an electrode of the ablation device into the lateral aspect of the thyroid nodule can be visualized. Typically, the electrode is positioned at the distal tip of the ablation device. Once the electrode is in position, a high level of energy is applied and tissue ablation occurs, which can be visualized by the clinician via the ultrasound system. Additionally, as tissue is ablated in response to heating, steam is generated which may result in audible “tissue popping” caused by the formation, expansion, and rupturing of bubbles within the heated tissue. The resulting “tissue popping” manifests as vibration of the ablation device, which is felt by the clinician that is holding the handle of the ablation device to indicate to the clinician that the tissue and/or electrode temperature is high and ablation is occurring at a high level. The resulting “popping” sounds may be loud enough to be heard by the clinician and the patient. For a conscious patient, hearing their own tissue “popping” as a result of bubbles expanding and exploding during an ablation procedure may be disconcerting to the patient. Typically, the clinician will attempt to minimize “tissue popping” by retracting the ablation device along the same path in which it was inserted while applying energy via the electrode, thereby creating a path of ablated tissue within the thyroid nodule. This approach is typically referred to as a “moving shot” technique. In conventional ablation techniques used for organs such as the liver, the electrode tip is fixed to the center of the target tumor, which is ablated by conducted heat. However, the thyroid gland is surrounded by several critical structures such as, for example, the esophagus, the trachea, the recurrent laryngeal nerve, the carotid artery, the vagus nerve, and the cervical sympathetic ganglion. Using the fixed technique, the nodule periphery and immediate surrounding tissue may be undertreated or overtreated, depending on the treatment time and energy applied. To avoid these shortcomings, a thyroid nodule may be treated using the “moving shot” technique by dividing the nodule into multiple conceptual ablation units, which can be treated individually using a high energy level. Since applying a high energy level ablates more tissue in less time, the clinician can control the pace of movement of the ablation device for a given electrode size to create a volume of ablated tissue relatively quickly while minimizing “tissue popping”.

Aside from the undesirable patient response to hearing “tissue popping” during an ablation procedure, the “tissue popping” may also be an indication of a loss of efficiency of energy delivery to target tissue since desiccation results in an increase in tissue impedance equaling a loss of energy delivery unless the generator accounts for that loss by delivering more power. Additionally, the “tissue popping” may create a void around the electrode as the tissue expands from the rupture caused by the steam, thereby resulting in a loss of tissue contact with the electrode and loss of energy delivery to the tissue.

The present disclosure is directed to an ablation system that provides feedback (e.g., audible, visual, etc.) during performance of an ablation procedure using the “moving shot” technique. The feedback is correlated with a number of Joules of energy delivered from an energy source to the ablation device over time during an ablation procedure. The number of Joules of energy delivered to the ablation device may be calculated, in real-time during an ablation procedure, based on parameters such as an impedance of the tissue being ablated at or near the electrode surface, a power level setting of the power source, and a procedure time (e.g., total time the energy source is set to deliver energy to the ablation device). A clinician may set a desired total number of Joules to be delivered to an ablation target and/or one or more desired total number of Joules of energy to be delivered to one or more corresponding portions of an ablation target at the energy source. The clinician may base the desired number of Joules of energy to be delivered on one or more parameters such as, for example, a size and/or dimensions of a nodule to be ablated. Once energy delivery from the energy source is initiated for an ablation procedure, the energy source initiates calculating the number of Joules of energy delivered to an electrode of the ablation device for ablating the ablation target. The energy source may display (e.g., via a display screen), in real-time during a procedure, the number of Joules delivered to the electrode of the ablation device as well as the set total number of Joules for ablating the entire ablation target and/or for ablating one or more portions of the ablation target. In aspects of the present disclosure, the feedback may be audible feedback and may include, for example, an intermittent or continuous audible sound (e.g., a beep, a tone, etc.) that increases in intensity, volume, frequency, periodicity, and/or tone as the number of Joules delivered approaches the set total number of Joules. Alternatively or additionally, a particular audible sound (e.g., a “ding”) may be broadcast to denote the number of Joules of energy delivered reaching the set total number of Joules. Alternatively or additionally, a particular audible sound may be broadcast prior to the number of Joules of energy delivered reaching the set total number of Joules to denote that the number of Joules of energy delivered is close to approaching the set total number of Joules. The audible feedback may also include a verbal indication (e.g., recorded or computer generated) providing information to the clinician about the number of Joules of energy delivered. For example, the verbal indication may be a Joule value, an indication that the number of Joules of energy delivered is approaching the set total number of Joules, and/or an indication that the number of Joules delivered has reached the set total number of Joules. The audible feedback may be provided via a suitable audio device (e.g., a speaker) that is either standalone or integrated with the energy source.

In aspects of the present disclosure, the feedback may be visual feedback provided, for example, via a display screen on the energy source. The visual feedback serves to provide a visual indication to the clinician of the proximity of the Joules delivered in relation to the set total number of Joules. For example, the displayed number of Joules delivered to the electrode of the ablation device may change in color as the number of Joules delivered approaches the set total number of Joules. In an aspect of the present disclosure, the displayed number of Joules delivered may initially be displayed in the color green. As the number of Joules delivered gets close (e.g., within a pre-determined threshold number of Joules) to the set total number of Joules, the displayed number of Joules delivered may change from being displayed in the color green to being displayed in the color yellow. When the number of Joules delivered reaches the set total number of Joules, the displayed number of Joules delivered may change from being displayed in the color yellow to being displayed in the color red. Additionally or alternatively, the displayed set total number of Joules may also change color similarly as described above with respect to the displayed number of Joules delivered. In aspects of the present disclosure, the feedback provided may be a combination of audible feedback and visual feedback.

In aspects of the present disclosure, the clinician may input at the energy source a size and/or dimensions of the ablation target (e.g., a thyroid nodule) to be ablated, in response to which, the energy source calculates and displays a suggested total number of Joules of energy to deliver to the ablation target for a complete ablation of the ablation target. The energy source may also calculate and display one or more suggested total number of Joules of energy to deliver to one or more corresponding portions of the ablation target for a complete ablation of those portions. Other input parameters may be used by the energy source to calculate the suggested total number of Joules of energy such as, for example, a size of the electrode of the ablation device to be used for the ablation procedure. In aspects of the present disclosure, the energy source may project the size and/or dimensions of an ablation volume based on the calculated number of Joules of energy being delivered, the procedure time, and the power setting of the energy source.

In some scenarios, if the clinician completes an ablation procedure without reaching the expected set total number of Joules, as can be determined by the audible feedback and/or the clinician viewing the displayed Joules of energy delivered relative to the set total number of Joules, the clinician may determine if additional ablation and/or procedure time is needed to complete the ablation of the ablation target. Likewise, if the audible feedback and/or the displayed Joules of energy delivered indicates that the set total number of Joules has been reached but the clinician has not completed the ablation procedure, the clinician may be prompted to evaluate the portion of the ablation target that has been ablated and/or take other action to improve the procedure.

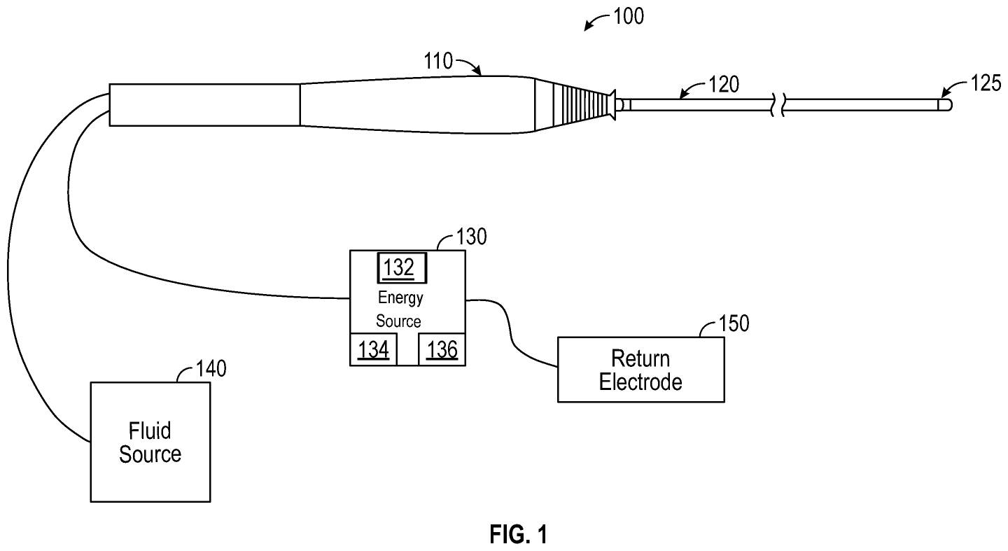

With reference to FIG. 1, an ablation system 10 according to aspects of the present disclosure is shown. The ablation system 10 generally includes an ablation device 100, an electrosurgical energy source, e.g., generator 130, and a fluid source 140. In aspects of the present disclosure, the ablation device 100 may be a monopolar ablation device utilizing an electrode 125 disposed on a distal portion of an elongated shaft 120 of the ablation device 100 to contact and ablate tissue. In this scenario, the ablation system 10 may include a remote return electrode 150 (e.g., return pad) coupled to the generator 130, as shown in FIG. 1, and configured to be adhered to a patient. In other aspects of the present disclosure, the ablation device 100 may be a bipolar ablation device utilizing two electrodes (not shown) disposed on the ablation device 100 to deliver energy to tissue disposed between the two electrodes. In this scenario, the two electrodes are positioned at a distal portion of the elongated shaft 120 of the ablation device 100 axially spaced (e.g., 5-10 mm) from one another and electrically isolated from one another. For example, an insulative material (not shown) may be disposed on the elongated shaft 120 between the two electrodes to electrically insulate the two electrodes from each other.

The fluid source 140 is configured to supply coolant fluid (e.g., via inflow fluid tubing) to the ablation device 100 to cool the elongated shaft 120 and/or the electrode 125 during an ablation procedure. Likewise, the ablation device 100 may be configured to return coolant fluid (e.g., via outflow fluid tubing) to the fluid source 140 and/or a suitable container. In the scenario where the ablation device 100 is configured as a monopolar ablation device, the return electrode 150 is electrically coupled to the generator 130 and, during an ablation procedure, the return electrode 150 is adhered to the skin of a patient for returning, to the generator 130, electrosurgical energy that is delivered to the patient via the ablation device 100. In the scenario where the ablation device 100 is configured as a bipolar device, one of the two electrodes serves the same purpose as the return electrode 150 of the above-noted monopolar configuration by returning, to the generator 130, energy delivered to tissue by the other of the two electrodes, thereby obviating the need for a remote return electrode adhered to the patient.

Commercially available ablation systems for use with the present disclosure include, for example, the Cool-tip™ RF Ablation System and the Accurian™ RF Ablation Platform both available from Medtronic plc of Dublin, Ireland. Although aspects of the present disclosure are described in terms of using RF energy to ablate tissue, such description should not be considered limiting. It is contemplated that the ablation device 100 and the generator 130 of the present disclosure may be configured for use with other suitable forms of ablation energy such as, for example, microwave, laser, ultrasonic, and/or cryogenics. In aspects of the present disclosure, the ablation device 100 and the generator 130 may be configured to use pulsed field ablation (PFA), which is a non-thermal method of ablating tissue using pulsed electric fields.

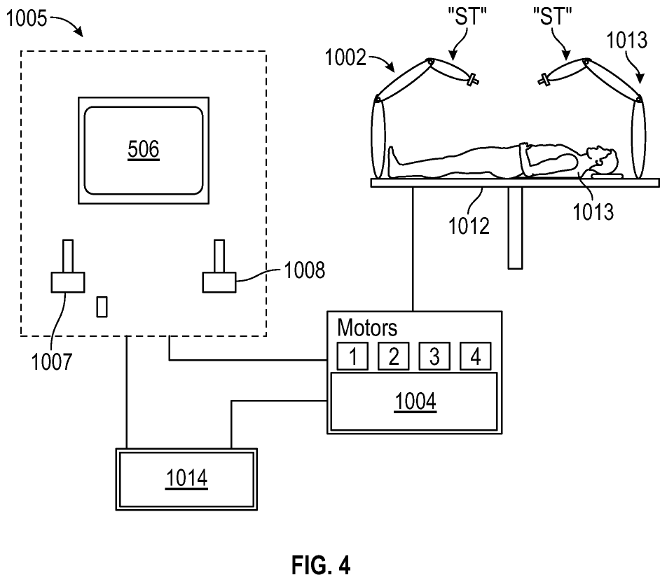

The ablation device 100 includes a handle 110 coupled to the elongated shaft 120. The handle 110 is configured to be gripped and manipulated by a clinician during an ablation procedure, although non-handle configurations are also contemplated, e.g., for mounting the ablation device 100 and/or attaching the ablation device 100 to a surgical robot arm (see FIG. 4). The electrode 125 is disposed at a distal end portion of the elongated shaft 120 and is configured for contacting and ablating tissue. The electrode 125 is configured to electrically couple to the generator 130 for providing electrosurgical energy (e.g., RF, microwave, laser, ultrasonic, cryogenic, etc.) to the electrode 125. The generator 130 may be configured to operate in one or both of a monopolar configuration or a bipolar configuration and include suitable outputs for delivering electrosurgical energy to the ablation device 100 and/or suitable inputs for returning electrosurgical energy to the generator 130. The generator 130 includes an impedance measurement circuit 136 configured to measure impedance of tissue being ablated by the electrode 125.

In some aspects of the present disclosure, the elongated shaft 120 of the ablation device 100 may include one or more sensors (not explicitly shown) configured to sense properties of surrounding tissue (e.g., tissue temperature, tissue impedance, etc.) and/or properties of the ablation device 100 (e.g., temperature of the elongated shaft 120 and/or the electrode 125). The one or more sensors may include, for example, a temperature-sensitive sensor (e.g., thermocouple, thermistor, etc.).

The generator 130 includes a display 132 configured to display, for example, a suggested total number of Joules, a set total number of Joules, a number of Joules of energy delivered, and/or a size of an ablation target (e.g., a thyroid nodule) to be ablated. The display 132 may display the suggested total number of Joules and/or the set total number of Joules for completing ablation of an entire ablation target and/or for completing ablation of a specific portion of the ablation target. The clinician may manually input the set total number of Joules based on the suggestion made by the generator 130 or may choose to manually input a set total number of Joules that is different than the suggestion made by the generator 130. The generator 130 may be configured to automatically set the total number of Joules based on the suggested total number of Joules. In aspects of the present disclosure, the display 132 may provide visual feedback correlated to a number of Joules delivered to the electrode 125 of the ablation device 100 for ablating tissue. For example, during a “moving shot” ablation procedure, the clinician may be alerted that the number of Joules of energy delivered to the electrode 125 is approaching a set total number of Joules and/or that the number of Joules of energy delivered to the electrode 125 has reached the set total number of Joules. In aspects of the present disclosure, the displayed number of Joules delivered to the electrode 125 of the ablation device 100 may change in color as the number of Joules delivered approaches the set total number of Joules. In one example approach, the displayed number of Joules delivered may initially be displayed in the color green. As the number of Joules delivered gets close (e.g., within a pre-determined threshold number of Joules) to the set total number of Joules, the displayed number of Joules delivered may change from being displayed in the color green to being displayed in the color yellow. When the number of Joules delivered reaches the set total number of Joules, the displayed number of Joules delivered may change from being displayed in the color yellow to being displayed in the color red. Additionally or alternatively, the displayed set total number of Joules may also change color similarly as described above with respect to the displayed number of Joules delivered.

The generator 130 may include an audio device 134 that provides audible feedback correlated to a number of Joules delivered to the electrode 125 of the ablation device 100 for ablating tissue. For example, during a “moving shot” ablation procedure, the clinician may be alerted that the number of Joules of energy delivered to the electrode 125 is approaching a set total number of Joules and/or that the number of Joules of energy delivered to the electrode 125 has reached the set total number of Joules. The audio device 134 may be, for example, a speaker that broadcasts an intermittent or continuous sound (e.g., a beep, a tone, or the like) that changes in volume, intensity, frequency, periodicity, and/or tone as the number of Joules of energy delivered changes relative to the set total number of Joules. In aspects of the present disclosure, the sound continuously or intermittently broadcast by the audio device 134 may be a verbal auditory signal that verbally indicates a numerical value of the number of Joules of energy delivered. For example, the audio device 134 may broadcast a recorded voice verbalizing a specific value of the number of Joules of energy delivered. With this feature in mind, the clinician may set a total number of Joules to be delivered during an ablation procedure (e.g., based on a given size of an ablation target and/or a given size of an electrode of the ablation device) such that the audio device 134 may, for example, verbally indicate the number of Joules of energy delivered when the number of Joules of energy delivered is approaching the set total number of Joules and verbally indicate the number of Joules of energy delivered has reached the set total number of Joules. Additionally or alternatively, the audio device 134 may simply provide a suitable verbal indication (e.g., a sound) that the number of Joules of energy delivered is approaching the set total number of Joules and/or that the number of Joules of energy delivered has reached the set total number of Joules. In an aspect of the present disclosure, the audio device 134 and/or the display 132 may be located on the ablation device 100 or the fluid source 140. The audio device 134 and/or the display 132 may also be a standalone device separate from the ablation device 100, the generator 130, and the fluid source 140.

In aspects of the present disclosure, the ablation system 10 may provide a combination of audible feedback via the audio device 134 and visual feedback via the display 132. In this scenario, the audible feedback and visual feedback may be correlated and/or synchronized with each other such that changes in the audible feedback are synched to changes in the visual feedback and vice versa.

In aspects of the present disclosure, the generator 130 may include one or more processors and one or more processor-readable media (e.g., memory) storing instructions. The instructions may be executed by the processor, which may include one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

In one or more examples, the described techniques may be implemented in hardware, software, firmware, or any combination thereof. If implemented in software, the functions may be stored as one or more instructions or code on a computer-readable medium and executed by a hardware-based processing unit. Processor-readable media may include non-transitory processor-readable media, which corresponds to a tangible medium such as data storage media (e.g., RAM, ROM, EEPROM, flash memory, or any other medium that can be used to store desired program code in the form of instructions or data structures and that can be accessed by a processor).

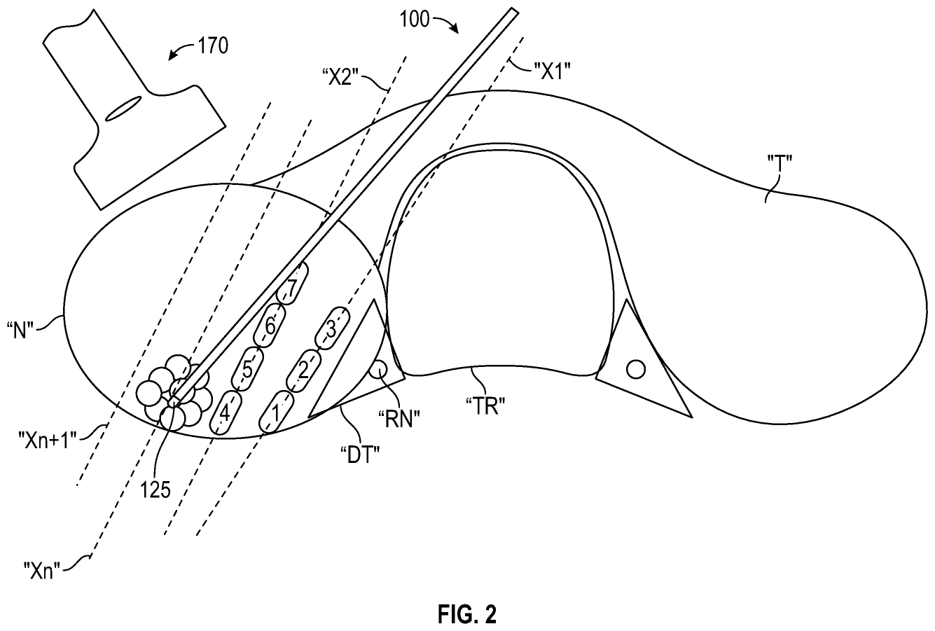

Referring now to FIG. 2, a depiction of a patient's thyroid gland “T” is shown in proximity to the patient's trachea “TR” and the “danger triangle” (referenced as “DT”) including the recurrent laryngeal nerve “RN”. The electrode 125 of the ablation device 100 is shown percutaneously inserted into a thyroid nodule “N” using the trans-isthmic approach under ultrasound guidance via an ultrasound imaging device 170. As would be understood, the ultrasound imaging device 170 is configured for coupling to a suitable display monitor (not shown) for visualizing, during an ablation procedure, the thyroid nodule “N” and the portion of the ablation device 100 percutaneously placed within the thyroid nodule “N”. Multiple conceptual ablation units are shown within the thyroid nodule and numbered (e.g., 1-7) in order to illustrate a standard “moving shot” technique for ablating the thyroid nodule “N”. The electrode 125 of the ablation device 100 is inserted through the isthmus such that the electrode 125 and the thyroid nodule “N” are visualized using the ultrasound imaging device 170. The electrode 125 is inserted into the thyroid nodule “N” and placed at the deepest portion of the thyroid nodule “N” at conceptual ablation unit “1” where RF energy is delivered to the thyroid nodule “N” via the electrode 125. While delivering energy to tissue, the clinician retracts the electrode 125 toward the most superficial portion of the thyroid nodule “N” in accordance with the order of numbering of each conceptual ablation unit. For example, the clinician will retract the electrode 125 along a first axis “X1” from conceptual ablation unit “1” to conceptual ablation unit “2” and subsequently to conceptual ablation unit “3” to ablate the thyroid nodule “N” unit-by-unit along the first axis “X1”. It should be understood that the clinician may move the electrode 125 slowly through the thyroid nodule “N” without stopping, or the clinician may at any time pause movement of the electrode 125 and reinitiate movement of the electrode 125. Following ablation of conceptual ablation unit “3”, the clinician will move the electrode 125 along a second axis “X2” adjacent to the first axis “X1” to once again place the electrode 125 at the deepest portion of the thyroid nodule “N” at conceptual ablation unit “4”, which is adjacent to conceptual ablation unit “1” at the deepest portion of the thyroid nodule “N”. In aspects of the present disclosure, energy delivery from the generator 130 to the electrode 125 may be interrupted during “forward” movement of the electrode 125 to place the electrode 125 at the deepest portion of the thyroid nodule “N”. For example, once ablation of the thyroid nodule “N” along the first axis “X1” is complete, energy delivery to the electrode 125 may be temporarily interrupted following ablation of conceptual ablation unit “3” and during insertion (e.g., “forward” movement along the second axis “X2”) of the electrode 125 to place the electrode 125 at the deepest portion of the thyroid nodule “N” at conceptual ablation unit “4”. Once the electrode 125 is placed at conceptual ablation unit “4”, RF energy is again delivered to the electrode 125 and the electrode 125 is retracted along the second axis “X2” unit-by-unit in accordance with the order of numbering of each conceptual ablation unit (e.g., from conceptual ablation unit “4” to conceptual ablatio unit “7”) until ablation of the thyroid nodule “N” along the second axis “X2” is complete. This “moving shot” technique is performed for subsequent adjacent axes (e.g., axis “Xn”, axis “Xn+1” and so on) until the thyroid nodule “N” is ablated, unit-by-unit and axis-by-axis, to completion. It is contemplated that the electrode 125 is not removed from the patient following completion of ablation along an axis and before moving the electrode 125 along a subsequent axis. For example, following ablation of conceptual ablation unit “3”, the clinician does not remove the electrode 125 from the patient before moving the electrode 125 along the second axis “X2” to place the electrode 125 at the deepest portion of the thyroid nodule “N” at conceptual ablation unit “4”. Rather, the clinician simply reinserts the electrode 125 into the thyroid nodule “N” while the electrode 125 is still inserted within the patient. It is also contemplated that the audio device 134 may continue to broadcast a sound during interruption of energy delivery to the electrode 125 so that the disclosed audible feedback feature will continue to operate when the clinician interrupts application of energy to tissue, moves the electrode 125, and reinitiates application of energy to tissue.

According to an aspect of the present disclosure, the audio device 134 provides audible feedback correlated to a number of Joules of energy delivered to the electrode 125 relative to a set total number of Joules during an ablation procedure performed using the “moving shot” technique. For example, the audio device 134 broadcasts a sound that will change in accordance with the number of Joules of energy delivered relative to the set total number of Joules. As the number of Joules of energy delivered increases, for example, the sound broadcast by the audio device 134 may increase in volume, intensity, frequency, periodicity, and/or tone as the set total number of Joules is approached. If the clinician completes an ablation procedure without reaching the expected set total number of Joules, as can be determined by the audible feedback and/or the clinician viewing the Joules of energy delivered on the display 132 of the generator 130, the clinician may determine if additional ablation and/or procedure time is needed to complete the ablation of the thyroid nodule. Conversely, if the audible feedback and/or the displayed Joules of energy delivered indicates that the set total number of Joules has been reached but the clinician has not completed the ablation procedure, the clinician may be prompted to evaluate the portion of the thyroid nodule that has been ablated and/or take other actions to improve the procedure.

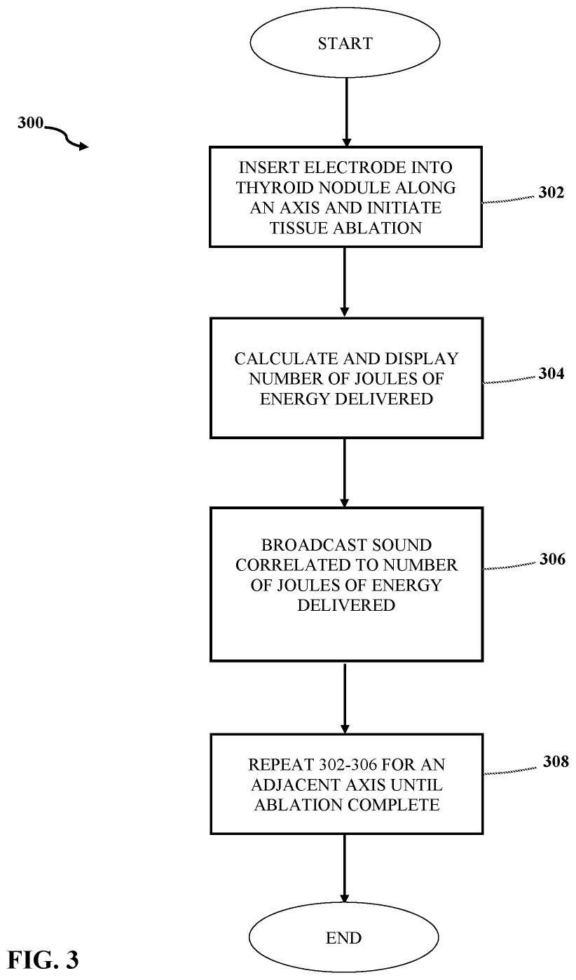

Turning now to FIG. 3, an example approach 300 is shown for performing an ablation procedure using a “moving shot” technique to ablate a thyroid nodule and providing audible feedback as a guide for ablating the thyroid nodule “N”. Although the example approach 300 is described in terms of ablating the thyroid nodule “N”, it should be appreciated that this procedure is merely provided as an example and that approach 300 may also be applied to other ablation targets/procedures (e.g., liver ablation, lung ablation, etc.) where using the “moving shot” technique may be practical. Moreover, although the example approach 300 is described in terms of providing audible feedback, it should be appreciated that providing audible feedback is provided as an example and that approach 300 may additionally or alternatively provide visual feedback as a guide for ablating the thyroid nodule “N”.

At block 302, the elongated shaft 120 of the ablation device 100 is percutaneously inserted, under ultrasound guidance, into the thyroid nodule “N” using a trans-isthmic approach such that the electrode 125 of the ablation device 100 is inserted into the thyroid nodule “N” and placed at the deepest portion of the thyroid nodule “N”. Once the electrode 125 is placed at the deepest portion of the thyroid nodule “N”, electrosurgical energy (e.g., RF energy) is delivered from the generator 130 to the electrode 125 to initiate ablation of the thyroid nodule “N”. Upon initiating power delivery, the generator 130 at block 304 initiates calculating and displaying the number of Joules of energy delivered to the electrode 125. Calculation of the number of Joules of energy delivered to the electrode 125 may be performed continuously during an ablation procedure and may be based on parameters such as, for example, a sensed property of the tissue being ablated (e.g., impedance), the procedure time, and/or the power level of energy delivered from the generator 130 to the ablation device 100. The display 132 of the generator 130 may display, in real-time during a procedure, the number of Joules of energy delivered to the electrode 125 as well as the set total number of Joules and/or the suggested total number of Joules. In aspects of the present disclosure, the generator 130 is configured to display one or both of a set total number of Joules corresponding to ablation of the thyroid nodule “N” along a particular axis (e.g., first axis “X1”) and a set total number of Joules corresponding to ablation of the entire thyroid nodule “N”. Thus, the clinician is able to monitor the number of Joules of energy delivered relative to the set total number of Joules while retracting the electrode 125 along each axis such that the clinician can consider whether additional ablation and/or ablation time is needed to complete ablation of a particular axis if the clinician is close to completing retraction of the electrode 125 along that axis but the number of Joules of energy delivered is not close to the set total number of Joules for that particular axis. Likewise, if the clinician is not close to completing retraction of the electrode 125 along a particular axis but the number of Joules of energy delivered is close to or at the set number of Joules for that axis, the clinician may consider evaluating the portion of the thyroid nodule “N” along that axis that has been ablated and/or consider increasing the pace of movement of the electrode 125 along that axis or along subsequent axes.

At block 306, the clinician retracts the electrode 125 from the deepest portion of the thyroid nodule along the first axis “X1” toward the most superficial portion of the thyroid nodule “N” while the audio device 134 intermittently or continuously broadcasts a sound that is correlated to the number of Joules of energy delivered to the electrode 125 such that the sound changes in response to a change in the number of Joules of energy delivered relative to the set total number of Joules. For example, as the number of Joules of energy delivered increases and approaches the set total number of Joules, the audible feedback may increase in intensity, volume, frequency, periodicity, and/or tone. In aspects of the present disclosure, a particular audible sound may be broadcast to denote the number of Joules of energy delivered reaching the set total number of Joules. Alternatively or additionally, a particular audible sound may be broadcast prior to the number of Joules of energy delivered reaching the set total number of Joules to denote that the number of Joules of energy delivered is close to approaching the set total number of Joules. The audible feedback may also include a verbal indication (e.g., recorded or computer generated) providing information to the clinician about the number of Joules of energy delivered. For example, the verbal indication may be a Joule value.

At block 308, once ablation of the thyroid nodule “N” along an axis (e.g., axis “X1”) is complete, the clinician will repeat blocks 302-306 for one or more adjacent axes (e.g., axis “X2”, “Xn”, “Xn+1”, etc.) until ablation of the thyroid nodule “N” is complete.

Turning now to FIG. 4, a robotic surgical system 1000 configured for use in accordance with the present disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a clinician, e.g., a clinician, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, the ablation device 100 of the present disclosure, thus providing any of the above-detailed functionality on a robotic surgical system 1000.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An ablation system, comprising:
an ablation device configured to be inserted into an ablation target, the ablation device having an electrode configured to be placed within the ablation target and retracted along an axis defined through the ablation target while delivering energy to tissue;
an electrosurgical generator configured to deliver electrosurgical energy to the ablation device and to calculate a number of Joules of electrosurgical energy delivered to the electrode;
a display device in communication with the electrosurgical generator and configured to display the calculated number of Joules of electrosurgical energy delivered to the electrode during an ablation procedure; and
an audio device in communication with the electrosurgical generator and configured to broadcast a sound correlated with the number of Joules of electrosurgical energy delivered to the electrode such that the audio device changes at least one property of the sound based on the calculated number of Joules of energy delivered to the electrode.

2. The ablation system according to claim 1, wherein the electrosurgical generator is configured to calculate a suggested total number of Joules of electrosurgical energy for delivering to the electrode based on at least one of a size of the ablation target or a size of the electrode.

3. The ablation system according to claim 2, wherein the display device is configured to display the suggested total number of Joules of electrosurgical energy for delivering to the electrode.

4. The ablation system according to claim 1, wherein the electrosurgical generator is configured to receive as input a total number of Joules of electrosurgical energy for delivering to the electrode.

5. The system according to claim 4, wherein the audio device is configured to broadcast a sound associated with at least one of:
the calculated number of Joules of electrosurgical energy delivered to the electrode reaching the total number of Joules of electrosurgical energy received as input; or
the calculated number of Joules of electrosurgical energy delivered to the electrode approaching the total number of Joules of electrosurgical energy received as input.

6. The ablation system according to claim 4, wherein the display device is configured to display the input total number of Joules of electrosurgical energy received by the electrosurgical generator.

7. The ablation system according to claim 1, wherein the electrosurgical generator is configured to receive as input at least one of a size of the ablation target or a size of the electrode, and to calculate a suggested total number of Joules of electrosurgical energy for delivering to the electrode based on the received input.

8. The ablation system according to claim 1, wherein the ablation target is a thyroid nodule.

9. The ablation system according to claim 1, wherein the audio device is integral with the electrosurgical generator.

10. The ablation system according to claim 1, wherein the display device is integral with the electrosurgical generator.

11. The ablation system according to claim 1, wherein the at least one property of the sound includes a volume, an intensity, a frequency, a periodicity, or a tone of the sound.

12. The ablation system according to claim 1, wherein the audio device is configured to broadcast a verbal indication associated with the calculated number of Joules of electrosurgical energy delivered to the electrode.

13. A method for ablating a thyroid nodule, the method comprising:
a) inserting an electrode of an ablation device into a first portion of a thyroid nodule along an axis defined through the thyroid nodule;
b) delivering electrosurgical energy from the electrode to the thyroid nodule while retracting the electrode along the axis toward a second portion of the thyroid nodule;
c) calculating a number of Joules of electrosurgical energy delivered to the electrode;
d) displaying the calculated number of Joules of electrosurgical energy delivered to the electrode;
e) broadcasting a sound correlated with the number of Joules of electrosurgical energy delivered to the electrode;
f) controlling at least one property of the sound based on the calculated number of Joules of electrosurgical energy delivered to the electrode; and
g) repeating the steps a) through f) for at least one additional axis defined through the thyroid nodule until the ablation procedure is complete.

14. The method according to claim 13, further comprising:
receiving as input at least one of a size of the thyroid nodule or a size of the electrode; and
calculating a suggested total number of Joules of electrosurgical energy for delivering to the electrode based on the received input.

15. The method according to claim 14, further comprising displaying the suggested total number of Joules of electrosurgical energy for delivering to the electrode.

16. The method according to claim 13, further comprising receiving as input a total number of Joules of electrosurgical energy for delivering to the electrode.

17. The method according to claim 16, further comprising broadcasting a sound associated with at least one of:
the calculated number of Joules of electrosurgical energy delivered to the electrode reaching the total number of Joules of electrosurgical energy received as input; or
the calculated number of Joules of electrosurgical energy delivered to the electrode approaching the total number of Joules of electrosurgical energy received as input.

18. The method according to claim 16, further comprising displaying the total number of Joules of electrosurgical energy received as input.

19. An ablation system, comprising:
an ablation device having an electrode configured to deliver electrosurgical energy to tissue;
an electrosurgical generator configured to deliver electrosurgical energy to the electrode and to calculate a number of Joules of electrosurgical energy delivered to the electrode; and
a feedback device in communication with the electrosurgical generator and configured to generate feedback correlated with the calculated number of Joules of electrosurgical energy delivered to the electrode relative to a set total number of Joules of electrosurgical energy.

20. The ablation system according to claim 19, wherein the feedback device is one of an audio device configured to generate audible feedback or a display device configured to generate visual feedback.
